(19) United States
(12) Patent Application Publication
(10) Pub. No.: US 2007/0249928 A1
(43) Pub. Date: Oct. 25, 2007

(54) Method and system for precise repositioning of regions of interest in longitudinal magnetic resonance imaging and spectroscopy exams

(75) Inventors: Daniel James Blezek, Niskayuna, NY (US); Ileana Hancu, Clifton Park, NY (US); Michelle Dumoulin, Philadelphia, PA (US); Richard Philip Mallozzi, Ballston Lake, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/590,949

(22) Filed: Nov. 1, 2006

Related U.S. Application Data
(60) Provisional application No. 60/793,058, filed on Apr. 19, 2006.

(51) Int. Cl. A61B 5/05

(52) U.S. Cl. 600/410

(57) ABSTRACT

The present invention provides a method and system for automatically processing longitudinal magnetic resonance (MR) images comprising obtaining a first MR image, selecting a region of interest within the first MR image, obtaining at least one subsequent MR image, applying a registration relative to the first MR image and the at least one subsequent MR image wherein the region of interest in the at least one subsequent MR image is repositioned analogously to the region of interest in the first MR image. A computer readable program is provided configured to apply a registration relative to a first MR image and an at least one subsequent MR wherein a region of interest in the at least one subsequent MR image is repositioned analogously to a region of interest in the first MR image.

### CROSS REFERENCE TO RELATED APPLICATIONS

This application is related to Provisional Application U.S. Ser. No. 60/793,058, entitled “Automatic Repositioning Of Single Voxels In Longitudinal 1H MRS Studies”, filed Apr. 19, 2006, the contents of which are herein incorporated by reference and the benefit of priority to which is claimed under 35 U.S.C. 119(e).

### BACKGROUND

The invention relates generally to magnetic resonance imaging and more particularly, to automatic registration of longitudinal scans to insure proper repositioning of regions of interest.

Proton magnetic resonance spectroscopy (MRS) studies following individuals over time in order to assess disease progression or response to therapy are becoming more and more common. Stringent requirements are generally imposed on reproducibility of data acquired in such longitudinal studies. In order for the effect of the disease or treatment to become apparent in the study, this effect needs to surpass the measurement error associated with the MRS data acquisition and quantification. Consequently, it is necessary to minimize measurement errors in data acquisition and quantification in MRS studies involving more than one scan for the same subject, referred to herein generally as “longitudinal studies.”

One of the sources of variability in longitudinal MRS arises from imperfect voxel re-localization in subsequent exams. Metabolite concentrations and relaxation times vary in different brain compartments (e.g. white matter, grey matter or cerebro-spinal fluid (CSF)) and also across anatomical brain regions. Thus, apparent changes in measured metabolite concentrations across sessions may appear only as a consequence of studying slightly different brain regions in follow-up exams. Typical mitigation of the voxel re-localization problem generally involves acquisition of high-resolution scout images, followed by careful prescription (by eye) of the MRS voxels using anatomical landmarks. This process requires skilled operator intervention for identification of anatomical landmarks and can be time-consuming, as occasionally double oblique scouts are needed to visualize the desired anatomical landmark. Additionally, this method does not lend itself to a careful a posteriori evaluation of the performance of the re-localization process for quality control.

A method to properly reposition MRS voxels in longitudinal exams has previously been reported (Hartman S L, Dawant B M, Parks M H, Schlack H, Martin P R, Comput Med Imaging Graph 1998; 22(6):453-461). This method involves operator selection of fiducials on the localizer MRI image. The registration process then computes a transformation that minimizes the residual error between the fiducials, and outputs coordinates of the center of the voxel that need to be prescribed in the follow-up exam. Besides requiring skilled operator intervention for fiducial extraction, this process is also time-intensive; the entire process of generating fiducials, registering, and generating the position of the new voxels of interest takes on the order of 10-15 minutes. The time interval needed for the registration procedure allows patient motion, creating a potential source of imprecision in voxel re-localization. Moreover, assuming a few degrees of rotation in each dimension between the baseline and follow up patient head positions, voxel overlap can never be maximized with this technique as much as it could if voxel rotations were also considered.

Other a posteriori automatic registration algorithms between baseline and follow-up localizer images from which CSI acquisitions are prescribed have been presented (Chu W-J, Pan J W, Hetherington H P, 2004; Kyoto. p. 105). CSI data is then selectively resampled and reconstructed, leading to dramatic improvements in the coefficients of variation for the studied metabolite concentrations. That procedure, however, does not allow for prospective acquisition of data from the same voxels, it only allows for a posteriori reconstruction. Possible rotation of the head between baseline and follow-up exams is not entirely accounted or corrected for.

What is needed is automatic registration of longitudinal MR images that allow one to acquire information from nearly identical regions of interest in a longitudinal exam, requiring no or substantially little user intervention.

### BRIEF DESCRIPTION

In a first aspect, the invention provides a method for processing longitudinal magnetic resonance (MR) images comprising obtaining at least a first MR image, selecting a region of interest within the at least first MR image, obtaining at least one subsequent MR image and applying a registration between first MR image and the at least one subsequent MR image wherein the region of interest in the at least one subsequent MR image is repositioned analogously to a corresponding region of interest in the first MR image.

Furthermore, in a second aspect the invention provides an imaging system for obtaining magnetic resonance images comprising a magnetic resonance imaging system adapted to obtain MRS images and a processor that is adapted to apply a registration relative to a first MR image and an at least one subsequent MR image wherein a region of interest in the at least one subsequent MR image is repositioned analogously to a region of interest in the first MR image.

Also, provided in a third aspect the invention provides an executable method for processing longitudinal magnetic resonance (MR) images comprising a computer readable program that is capable of applying a registration relative to a first MR image and an at least one subsequent MR image wherein a region of interest in the at least one subsequent MR image is repositioned analogously to a region of interest in the first MR image and a storage data disc for storing data on a storage medium.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of a Magnetic Resonance Imaging system to which embodiments of the present invention are applicable.

FIG. 2 is a diagram depicting two choices for protocols for the longitudinal exam, comparing automatic to eye voxel repositioning procedure.

FIG. 3 is an illustration of voxel locations in a several scans using different voxel re-localization protocols.

### DETAILED DESCRIPTION

The following detailed description is exemplary and not intended to limit the invention of the application and uses of the invention. Furthermore, there is no intention to be limited by any theory presented in the preceding background of the invention of the following detailed description of the drawings.

In a first embodiment, the invention provides a method for processing longitudinal magnetic resonance (MR) images comprising obtaining at least a first of MR images, selecting a region of interest within the first set of MR images, obtaining at least one subsequent MR image, and applying a registration relative to the first MR image and the at least one subsequent MR image wherein the region of interest in the at least one subsequent MR image is repositioned analogously to the region of interest in the first MR image. As used herein, the term “region of interest” refers to at least one voxel, or more specifically a chosen anatomical region of interest located inside the at least one voxel, which is to be chosen by the operator. Furthermore, a region of interest may comprise a metabolic region of interest, inside of which the concentration of metabolites such as glutamate or choline may be measured through a magnetic resonance spectroscopy exam. The term “region of interest overlap” refers to the percentage of the first region of interest voxel volume encompassed by the longitudinal voxel. As used herein the term “registration” refers to the correlation of two separate image regions or volumes relative to one another. For instance, registration of a first scan and any subsequent longitudinal scans. As used herein the term “analogous” or “analogously” refers to the occurrence or situation wherein the chosen regions of interest in the first and subsequent MR images are positioned to be spatially aligned in 3D (i.e. registered on all 3 Cartesian axes) with respect to one another.

In a second embodiment, the invention provides an imaging system adapted to obtain MRS images and a processor that is adapted to apply a registration relative to a first MR image and an at least one subsequent MR wherein a region of interest in the at least one subsequent MR image is repositioned analogously to a region of interest in the first MR image.

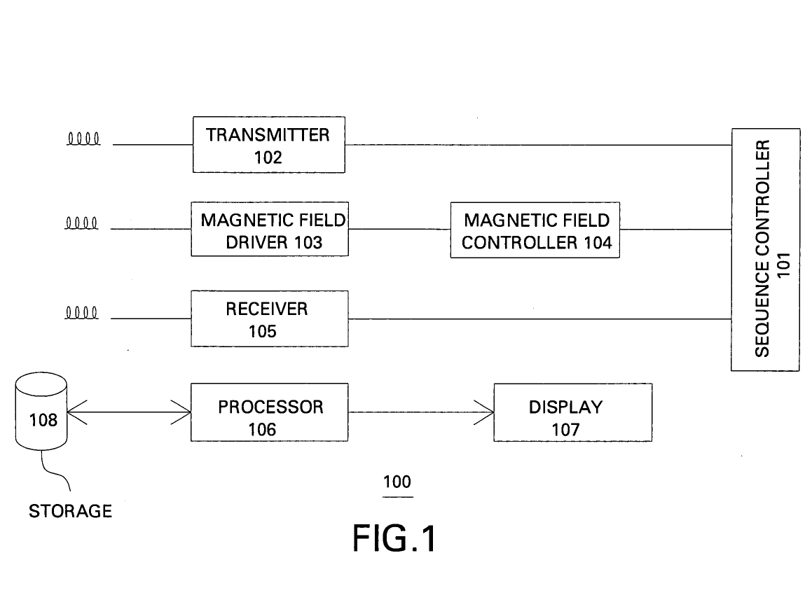

Referring to FIG. 1, there is shown a block diagram of a magnetic resonance imaging (MRI) system for which embodiments of the present invention are applicable. The MRI system 100 comprises a sequence controller 101 for controlling various components of the system, as is well-known, for detecting magnetic resonance signals from the part of an object being imaged; a transmitter 102 for generating an radio frequency (RF) pulse to cause resonance; a magnetic field driver 103 for driving a field gradient in a known manner; a magnetic field controller 104 for controlling the magnetic field; a receiver 105 for receiving and detecting magnetic resonance signals generated from the object; a processor 106 for performing image reconstruction and various calculations for system operation; a display 107 for displaying images; and a peripheral memory device 108 for storing detected signal data and reconstructed k-space data.

In a well-known manner, processor 106 is configured such that there is sufficient memory for storing measured data and reconstructed images. The memory is sufficient to store the whole of N-dimensional measured data as well as reconstructed data. Also in a well-known manner, a MR image is constructed from the image or k-space data corresponding to a predetermined plurality of applications of a MRI pulse sequence initiated by a RF pulse such as from transmitter 102 of FIG. 1. The image is updated by collecting image or k-space data from repetitive MRI pulse sequences. An MR image is reconstructed by performing a series of Fourier transforms along a set of orthogonal directions in k-space. As used herein, “adapted to”, “configured” and the like refer to operation capabilities of electrical elements such as analog or digital computers or application specific devices (such as an application specific integrated circuit (ASIC)) that are programmed to perform a sequel to provide an output in response to given input signals.

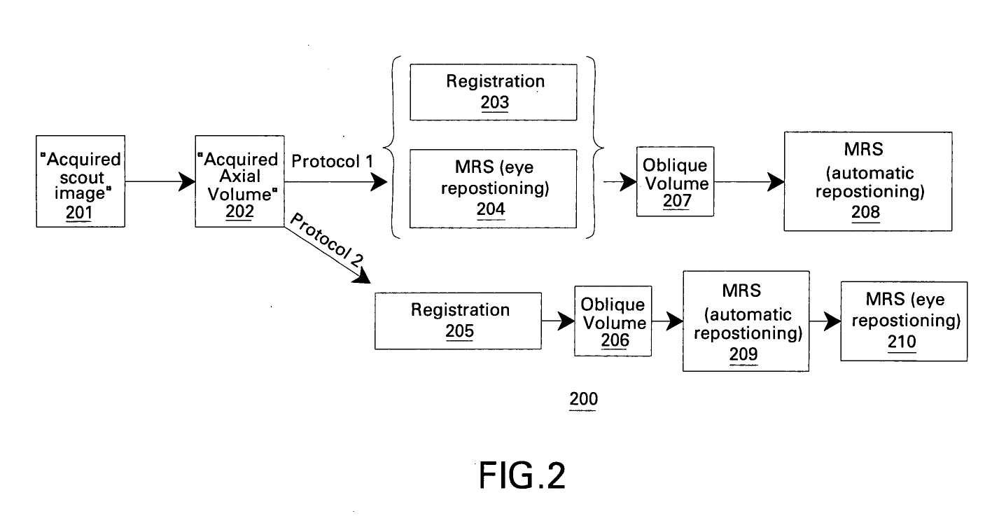

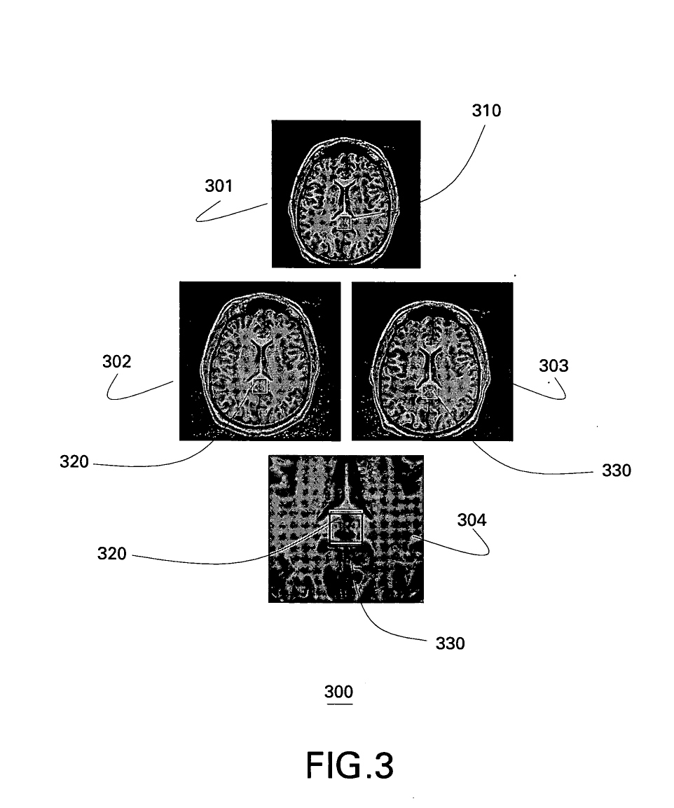

In embodiments of the present invention, processor 106 is configured to process longitudinal MR images, which will be described in greater detail is FIGS. 2-3.

As a general description, magnetic resonance imaging (MRI) is a well-known imaging method in which magnetic moments are excited at specific nuclear spin precession frequencies that are proportional to the magnetic field occurring within the magnet of the MRI system. Spin is a fundamental property of nature, such as electrical charge or mass. Precession is a rotational motion about an axis of a vector whose origin is fixed at the origin. The radio-frequency (RF) signals resulting from the precession of these spins are received typically using RF coils in the MRI system and are used to generate images of a volume of interest. A pulse sequence is a selected series of RF pulses and/or magnetic field gradients applied to a spin system to produce a signal representative of some property of the spin system.

In addition, an MRI system may be adapted to obtain magnetic resonance spectroscopy (MRS) images, to obtain metabolic information (e.g. metabolite concentrations) from the anatomical regions of interest. Similar to nuclear magnetic resonance (NMR), different molecules have different signatures in the NMR spectrum, and the concentration of different molecules of interest can be quantified from their NMR spectrum. This technique takes advantage of the magnetic spin properties of biological samples to reveal metabolic information.

In a well-known manner for tracking disease progression and monitoring changes in a particular anatomy due to disease, longitudinal images may be used. As used herein, “longitudinal images” means one or more subsequent images subsequent to a baseline scan, of a single subject or a plurality of subjects, relating to a first image of a single subject or a plurality of subjects. For instance, within a cohort of subjects diagnosed with the same disease, the baseline scan of single subject within the cohort may be used as the baseline scan for all other subjects within the cohort. The longitudinal images for each subject thus may be related back to the single subjects baseline scan. In principle however, the more spatially congruent each longitudinal image is as it relates to the first image, the more accurate the interpretation of each image will be. Also, longitudinal images may be employed to track the progression of disease for other conditions and that longitudinal images may be employed to monitor response (favorable or unfavorable) to treatment.

To obtain a first MR image, also referred to herein as a baseline scan, transmitter 102 of FIG. 1 is desirably adapted to generate and apply, during MR imaging, a MRI pulse sequence to obtain MRI images. It is to be appreciated that field strengths between 0.5 T and 18 T may be used. In an exemplary embodiment, during the baseline scan for each subject using a 1.5 T MRI system, the pulse sequence employs a fast localizer followed by the acquisition of a whole brain volume (axial orientation), using a three-dimensional fast spoiled gradient echo sequence (3D FSPGR) with inversion recovery preparation (inversion time=300 ms). Furthermore, acquiring volumes may comprise using x/y/z resolutions of 1.15 mm/1.15 mm/2 mm. Also, a PRESS sequence may be used with an echo time (TE) of 35 ms, and a repetition time (TR) of 200000 ms to obtain whole brain MRS scans. Automated optimization of transmitter pulse power, water suppression and gradient shimming, should consistently yield spectra with linewidths of about 6-7 Hz.

To obtain a subsequent brain scan of a single subject, also referred to herein as a longitudinal scan, transmitter 102 is desirably adapted to generate and apply, during MR imaging, a pulse sequence used to acquire the first or baseline image to obtain at least one subsequent MRI image. Referring to FIG. 2, there is shown a more detailed flow diagram of a method for obtaining longitudinal images and comparing the performance of an eye voxel repositioning procedure and automatic repositioning procedure. Two protocols are shown, to alternate the order of the acquisition of the eye and automatic localization procedures in order not to compound potential motion artifacts on either the eye or automatic procedure. Protocol 1 comprises acquiring a scout image 201, followed by acquiring an axial volume 202. Performing registration 203 and MRS eye repositioning 204 occurs simultaneously. An oblique volume 207 is then acquired followed by an MRS image 208. More specifically, the oblique volume 207 is acquired using a 3D fast-spoiled gradient echo (FSPGR) sequence accommodating as input the six parameters output by the registration algorithm (three translations and three rotations). The prescription of an oblique scan, tilted on all three physical axes, may be accomplished by manual input of the three Euler angles (defined as floating point user variables). Protocol 2 comprises acquiring a scout image 201, followed by acquiring an axial volume 202. Performing registration 205, then acquiring an oblique volume 206, followed by automatic repositioning 209 and eye repositioning 210. Registration step 205 will be described in greater detail below and with reference to FIG. 3.

To locate at least one region of interest, the operator may choose an anatomical region of the brain (e.g. hippocampus, or caudate nucleus) from which he/she desires metabolic information (e.g. choline concentration). Furthermore, the operator may choose to acquire the metabolic information of interest using MRS. Referring to FIG. 3, an embodiment for locating a metabolic region of interest as in 301 is provided. In this embodiment, an operator may place a 2×2×2 cm voxel (shown as 310) to enclose a region of interest in the image, from which metabolic information of interest is then acquired through a MRS acquisition. This region can be, for example, the posterior cingulate gyrus of a subject. However, it is also to be appreciated that voxel 310 may be of any size, or a group of voxels may be placed at any chosen location within the subject scan. The metabolite of interest should be generally associated with a disease condition, where the images or MRS spectra are used to track the disease or monitor response to therapy for the disease. Metabolic regions of interest may contain, but are not limited to, metabolites such as choline, NAA, mI, Cr, and any combination thereof. In addition, each metabolite may be quantified using spectra, and version 6.0 of LCModel, commercially available from LCModel, with fitting performed between 0 and 4 ppm using the basis set supplied by the program's vendor.

Referring further to FIG. 3, locating a corresponding metabolic region of interest is accomplished by eye repositioning voxel 320 as shown in image 302 or by automatic repositioning voxel 330 as shown in image 303. A composite image 304 shows each repositioning method allowing for a quantitative comparison of the accuracy of each repositioning as it relates to the baseline image, in other words the correlation of the eye positioned voxel 320 vs. the correlation of the automatic repositioned voxel 330 relative to the voxel 310 of the original or baseline image. It was found using the results from four volunteer images that the automatic repositioning using the registration algorithm described below produced a more accurate voxel overlap than did the eye repositioning overlap, as shown in Table 1 below.

TABLE 1

| Volunteer | 1 | 2 | 3 | 4 | Average |
|---|---|---|---|---|---|
| Eye repositioning overlap | 84 ± 14 | 86 ± 7 | 86 ± 7 | 87 ± 5 | 86 ± 9 |
| [%] | | | | | |
| Automatic repositioning | 94 ± 2 | 94 ± 2 | 93 ± 2 | 94 ± 3 | 94 ± 2 |
| overlap [%] | | | | | |

To precisely locate the metabolic region of interest in a follow-up scan, a registration procedure is generally needed. Generally, image registration is the process of aligning two images or image volumes. In its most basic form, a registration algorithm optimizes a metric function by adjusting the parameters of a transformation mapping one image to the other. It may adjust for orientation changes, transverse changes, longitudinal changes, rotational changes, or any combination thereof. More specifically, in embodiments of the present invention, the processor 106 of FIG. 1 is adapted to perform registration according to the following: The Mutual Information metric may be used, which is a measure of information one signal source provides about another. The two information sources are images; when aligned, the two images will provide maximal information about each other. To construct the mutual information of two imaging volumes, denoted A and B, the entropy of imaging volume A, whose pixel values, aᵢ, are considered to be random variables, is first defined as

$$H ( A ) = - {\sum }_{i = 1}^{M} p ( {a}_{i} ) log ( p ( {a}_{i} ) ) \qquad ( 1 )$$

Here log( ) represents the natural logarithm of a number, p(aᵢ) is the likelihood of finding pixels with intensities aᵢ throughout the imaging volume, and M represents the number of bins in which image intensity has been partitioned into. A digital image whose pixels are encoded in N bits, can have 2^{N} different grayscale values in a pixel and therefore the maximum value of M can be 2^{N}. In practice, however, choosing the maximum value for M would lead to computationally intense calculations, particularly when calculating the joint entropy of two images; M=30 is a good compromise between precision and computational cost.

The joint entropy of imaging volumes A and B, H(A,B) is then defined as:

$$H ( A , B ) = - {\sum }_{i = 1}^{M} {\sum }_{j = 1}^{M} p ( {a}_{i} , {b}_{j} ) log ( p ( {a}_{i} , {b}_{j} ) ) \qquad ( 2 )$$

Here p(aᵢ,bⱼ) is the likelihood of finding pixels with intensities aᵢ in image A at the same time that the corresponding pixel in image B has intensity bⱼ. Given the similarities between the two image sets to be registered, one could partition both image intensities in the same number of bins, ie M=30. The mutual information of two imaging volumes A and B, MI(A,B), is defined as

MI(A,B)=H(A)+H(B)−H(A,B) (3)

We are searching for the transformation T, that maximizes the mutual information defined as

MI(B,T(F))=H(B)+H(T(F)))−H(B,T(F)) (4)

Here B is the baseline imaging volume, F is the follow up imaging volume, and T(F) is the transformed follow up imaging volume. Because the registration may be intra-subject with relatively short times between scanning sessions, i.e. no significant changes in morphology, a six degrees of freedom rigid transformation may be used. If B=f₁(r) as the baseline imaging volume (where r=[x, y, z]^{T} is a position vector variable pointing to the space variable (x,y,z)), then the transformed follow-up imaging volume, T(F) can be expressed as T(F)=f₂(r)=f₁(Rr+t). Here R is a 3×3 orthonormal matrix with a determinant of 1, fully characterized by three rotation parameters (Euler angles), and t is a 3×1 translation vector comprised of three translational parameters. The matrix R and vector t represent the “motion” from time point 1 to time points 2. The objective of the registration algorithm is to estimate, as accurately as possible, the six parameters that specify R and t, by optimizing the mutual information cost function. A conjugate gradient descent method (15) may be used to search for the transformation that maximizes the mutual information MI(B, T(F)).

In a further embodiment, processor 106 is adapted to automatically measure quantitative changes between baseline scans and longitudinal scans, specifically in reference to each corresponding region of interest. Additionally, processor 106 is adapted to automatically send detailed exam information to display 107 and storage 108.

In an exemplary embodiment, a longitudinal exam protocol started with a short localizer, followed by an 86 slice axial brain volume with parameters identical acquisition to the baseline scan. Two types of protocols were used to compare the eye and automatic repositioning procedure for the regions of interest. In the first protocol, the MRS data was obtained by repositioning a voxel on the axial volume as close as possible to the initial voxel location; for each subject, screen shots showing the baseline voxel prescription were available to the operator. During the image acquisition, registration was performed between the first axial scan and the longitudinal axial scan yielding the rigid transformation that connected the head positions in the two exams. An oblique volume was then acquired in the follow up exam using the parameters output by the registration algorithm had identical acquisition parameters and brain coverage as the first exam. The images in the oblique volume were then used as support for the acquisition of another spectrum from a voxel whose positioning was given by the registration algorithm (automatic voxel repositioning procedure). The algorithm applied the inverse transformation to the position of the initial voxel location, thus mapping the first metabolic region of interest to the same anatomic location in the longitudinal scan.

In a second protocol to compare the eye and automatic repositioning procedures, the axial volumes were acquired. They were then followed by the acquisition of the oblique volume with identical orientation as the first exam. A spectrum was acquired from a voxel whose location was dictated by the output of the registration procedure. Following the completion of the first MRS exam, a longitudinal MRS scan was then prescribed on the axial series; the location of this last voxel, repositioned by eye, was chosen to match the location of the first metabolic region of interest as well as possible. These two protocols were alternated for subjects, in order to insure that the same amount of time elapsed between the automatic and eye repositioning of the voxel and the localizer scan.

Additionally, each metabolite of interest may be quantified, as shown by example in table 2. Here, the coefficient of variation (CV) and the Pearson correlation coefficients (r) are displayed for the volunteers studied, for both the eye repositioning and the automatic repositioning procedures.

TABLE 2

| | | Cr | Glu | Glu/Cr | MI | mI/Cr | Cho | Cho/Cr | NAA | NAA/Cr |
|---|---|---|---|---|---|---|---|---|---|---|
| CV | Eye | 3.84 | 7.40 | 6.96 | 6.18 | 5.7 | 4.53 | 5.33 | 4.32 | 3.65 |
| [%] | repositioning | | | | | | | | | |
| | Automatic | 2.76 | 6.23 | 6.89 | 6.01 | 5.15 | 4.82 | 4.73 | 4.22 | 4.12 |
| | repositioning | | | | | | | | | |
| r | Eye | 0.68* | | | 0.85** | 0.78** | 0.96** | 0.87** | 0.82** | 0.46⁺ |
| | repositioning | | | | | | | | | |
| | Automatic | 0.93** | | | 0.87** | 0.82** | 0.95** | 0.92** | 0.9** | 0.76** |
| | repositioning | | | | | | | | | |

In this particular exemplary embodiment, each metabolite was quantified using a linear combination of basis spectra, and version 6.0 of LCModel. Fitting was performed between 0 and 4 ppm using the basis set supplied by the program developer. NAA concentrations included the sum of the concentrations of NAA and NAA-glutamate. Similarly, choline (Cho) concentrations include the sum of all the Cho containing compounds. Additional metabolite concentrations that may be present include creatine (Cr), myo-Inositol (ml) and glutamate (Glu). Absolute quantitation was based on the reciprocity principle and signal calibration was performed with a 50 mM NAA phantom scan prior to the scans.

Referring further to table 2, the average intra-day, intra-volunteer coefficients of variation (CV's) for the metabolites recorded are shown. Consistent decreases in CV's can be noted when the automatic re-localization procedure was used, as seven out of the nine metabolites studied showed decreased CV's. More precisely, three out of nine metabolites showed consistent improvements (>10%) in CV's (Cr, Glu and Cho/Cr), while only one showed decreased (>10%) repeatability (NAA/Cr). Out of the rest of five metabolites, four showed marginal improvement (0-10%) in CV's (Glu/Cr, mI, mI/Cr, NAA), while only one other metabolite concentration showed marginally decreased (0-10%) repeatability (Cho). While both repositioning methods generally offer acceptable short term reproducibility coefficients (defined as Pearson correlation coefficients higher than 0.75) for all metabolite concentrations and concentration ratios studied (except for Glu and Glu/Cr), increased reproducibility coefficients can be noted when the automatic repositioning procedure is used. Most notably, the reproducibility becomes borderline to unacceptable for Cr and NAA/Cr when the manual repositioning technique is used.

The registration algorithm presented does not have any stringent requirements with respect to image contrast or brain coverage. For instance, in an exemplary embodiment, two sets of images were acquired with one volunteer in one position. The first set of images covered the whole brain (86 slices in axial orientation), had high resolution, (1.15 mm ×1.15 mm ×2 mm), and high contrast (inversion recovery preparation was used, with an inversion time of 300 ms). The second set of 18 images covered about a quarter brain, had low resolution (1.87 mm ×1.87 mm ×2 mm), and low contrast (no inversion recovery preparation). The volunteer moved to a new position and the same acquisition was performed. To estimate the accuracy of the algorithm, the parameters calculated by registering the high-resolution volumes were compared with the parameters calculated by registering the low-resolution volumes. The difference in parameters reported by the two registration operations were [0.33°, 0.22°, 0.18°] for the three Euler angles, and [0.22 mm, 0.18 mm, −0.19 mm] for the three displacements.

Additionally, by using this implementation, images with different contrast can be registered, as well as images covering different brain regions (but with significant overlap). For example, a full brain, T₁ weighted data set from a baseline scan was successfully registered with a quarter brain, T₂ weighted data set in the follow-up exam. In this instance, subjective evaluation was performed by visual inspection. This additional flexibility might be needed when two different protocols are selected for the baseline and follow-up exams. Moreover, the first image of a metabolic region of interest may be compared to the metabolic region of interest in each subsequent longitudinal image. The operator may compare each images optically, and track metabolic regions of interest over time. Furthermore, the operator can track disease progression or response to therapy by tracking, for example, lesion growth or reduction.

In another exemplary embodiment, four healthy, normal volunteers were scanned on four different days during the course of six months, three times each day. The subjects were then removed from the scanner room between the daily scanning sessions. Care was taken for consistent repositioning of the subjects in repeat scans. By moving the subject's head, the longitudinal landmark line was aligned with the subject nose, while the transverse landmark light was aligned with the inter-pupillary line. If slight rotations were discovered following the acquisition of the scout, however, no subject repositioning in the magnet was performed. To assess the performance of the registration algorithm, one of the high-resolution baseline volumes for one of the volunteers was rotated and translated by a known amount, with rotation angles limited to ±8 degrees and translation limited to ±10 mm in all dimensions. The baseline volume was then registered to the transformed volume, and the errors (defined as the difference between the known and calculated parameters) for the three translations and three rotations recorded. The average realignment error (for all voxels) was also computed as

$$ɛ = \frac{1}{N} {\sum }_{i = 1}^{N} {‖ {r}_{i} - {\hat{R}}^{- 1} ( {Rr}_{i} - t - \hat{t} ) ‖}_{2} \qquad ( 5 )$$

Here N represents the number of voxels in the imaging volume, R and t are the known rotation and translation matrices, respectively, \hat{R} and \hat{t} are their estimates obtained from the registration algorithm. Note that, if \hat{R}=R and \hat{t}=t, then the realignment error becomes zero. This process was repeated 100 times, by randomly choosing a set of rotation and translation parameters bounded by the limits specified above.

In the same exemplary embodiment, metabolic region of interest overlap calculations were measured for both automatic and eye repositioning. The longitudinal axial was registered to the first metabolic region of interest axial using the algorithm described above; the longitudinal voxel (placed by eye) was transformed into the space of the baseline scan. Samples, regularly spaced at 0.1 mm intervals inside the baseline voxel were compared against the transformed follow up voxel. Samples were considered overlapping if they are contained in both the first metabolic region of interest voxel and the transformed longitudinal voxel. The procedure was repeated for the triple oblique exam and automatically placed voxel.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for processing longitudinal magnetic resonance (MR) images comprising:
obtaining at least a first MR image;
selecting a region of interest within the at least first MR image;
obtaining at least one subsequent MR image;
applying a registration relative to the first MR image and the at least one subsequent MR image wherein the region of interest in the at least one subsequent MR image is repositioned analogously to a corresponding region of interest in the at least first MR image.

2. The method of claim 1, wherein the obtaining a first MR image step and the obtaining at least one subsequent MR image step are acquired using a magnetic resonance imaging (MRI) system.

3. The method of claim 1, wherein the region of interest is an anatomical region of interest.

4. The method of claim 1, wherein the region of interest is a metabolic region of interest and is probed through a magnetic resonance spectroscopy acquisition.

5. The method of claim 1, wherein the registration generates adjustments of the imaging planes for different positioning of a subject between the first and the at least one subsequent image.

6. The method of claim 1 wherein longitudinal M images are acquired for observing the progression of a disease or response to treatment of the disease associated with the region of interest.

7. The method of claim 6, further comprising displaying the first and the at least one subsequent images for use in comparing the images for changes between each corresponding region of interest.

8. The method of claim 6, further comprising automatically measuring quantitative changes between the first region of interest and each corresponding region of interest by the use of the processor.

9. An imaging system for obtaining magnetic resonance images comprising:
a magnetic resonance imaging system adapted to obtain MRS images;
a processor that is adapted to apply a registration relative to a first MR image and an at least one subsequent MR wherein a region of interest in the at least one subsequent MR image is repositioned analogously to a region of interest in the first MR image.

10. The system of claim 9, wherein a result of the registration is automatically communicated from the processor to the imaging system to obtain images for comparison.

11. The system of claim 9, wherein the processor automatically measures quantitatively the changes between the first region of interest and each corresponding region of interest.

12. The system of claim 9, wherein the images are used to track disease progression and monitor response to therapy.

13. An executable method for processing longitudinal magnetic resonance (MR) images comprising:
a computer readable program that is capable of applying a registration relative to a first MR image and an at least one subsequent MR wherein a region of interest in the at least one subsequent MR image is repositioned analogously to a region of interest in the first MR image;
a storage data disc for storing data on a storage medium.
